(19) United States
(12) Patent Application Publication
(10) Pub. No.: US 2014/0148358 A1
(43) Pub. Date: May 29, 2014

(54) SYSTEM AND METHOD FOR DETECTING ONE OR MORE ANALYTES IN A FLUID

(71) Applicants: Fong Yau Sam Li, Singapore (SG); Huanan Wu, Singapore (SG); Mahe Liu, Singapore (SG)

(72) Inventors: Fong Yau Sam Li, Singapore (SG); Huanan Wu, Singapore (SG); Mahe Liu, Singapore (SG)

(73) Assignee: NATIONAL UNIVERSITY OF SINGAPORE, Singpore (SG)

(21) Appl. No.: 14/130,464

(22) PCT Filed: Jun. 28, 2012
(86) PCT No.: PCT/SG2012/000231

§ 371 Date: Dec. 31, 2013

Related U.S. Application Data
(60) Provisional application No. 61/503,690, filed on Jul. 1, 2011.

(51) Int. Cl. G01N 33/18; G01N 33/543; G01N 1/18

(52) U.S. Cl. CPC G01N 33/18; G01N 1/18; G01N 33/54373
USPC 506/9; 204/409; 436/501; 435/7.1; 435/5; 205/775; 506/39; 422/69; 422/82

(57) ABSTRACT

Various embodiments provide a system for detecting one or more analytes in a fluid. The system comprises: a controller adapted to obtain one or more sample streams of the fluid. The controller is configured in use to form a plurality of output streams. Each output stream comprises at least part of one of the one or more sample streams. The system further comprises: a detector adapted to receive from the controller the plurality of output streams and comprising a plurality of sensors. Each sensor is operable to detect an interaction between a corresponding detecting agent and a corresponding analyte. The detector is configured in use to detect one or more said interactions using the plurality of output streams to determine if the fluid contains one or more said analytes. A corresponding method is also provided.

### TECHNICAL FIELD

Various embodiments relate to a system and method for detecting one or more analytes in a fluid.

### BACKGROUND

It is known to detect one or more analytes in a fluid. For example, where the fluid is water, it is known to detect the presence of one or more analytes in order to establish the quality of the water. Establishing water quality can be an important precursor to determining if the water is fit for human consumption.

Analysis of fluid quality parameters is usually performed off-line and using wet chemistry methods. These methods can take hours or days to complete, require skilled personnel and involve the use of toxic chemicals. Accordingly, it is desirable to develop automated systems and methods which can be performed in-line with the fluid system and do not require use of toxic chemicals.

### SUMMARY

Various embodiments provide a system for detecting one or more analytes in a fluid, the system comprising: a controller adapted to obtain one or more sample streams of the fluid, the controller being configured in use to form a plurality of output streams, each output stream comprising at least part of one of the one or more sample streams; and a detector adapted to receive from the controller the plurality of output streams and comprising a plurality of sensors, each sensor being operable to detect an interaction between a corresponding detecting agent and a corresponding analyte, the detector being configured in use to detect one or more said interactions using the plurality of output streams to determine if the fluid contains one or more said analytes.

In an embodiment, the corresponding detecting agent of one of the plurality of sensors is formed on said one sensor.

In an embodiment, the interaction detected by said one sensor comprises binding of the corresponding analyte to the corresponding detecting agent, and wherein the controller is adapted to obtain a flushing fluid configured in use to remove a bound analyte, the controller being configured in use to provide the flushing fluid to the detector to remove the bound corresponding analyte from said one sensor.

In an embodiment, the controller is adapted to obtain a regeneration fluid configured in use to form a detecting agent when exposed to a sensor, the controller being configured in use to provide the regeneration fluid to the detector to form the corresponding detecting agent of one of the plurality of sensors by exposing said one sensor to the regeneration fluid.

In an embodiment, the regeneration fluid comprises the corresponding detecting agent and exposure of said one sensor to the regeneration fluid transfers the corresponding detecting agent to said one sensor.

In an embodiment, the system further comprises: a regeneration fluid reservoir adapted to temporarily store the regeneration fluid before the controller obtains the regeneration fluid, the regeneration fluid reservoir being configured in use to control the temperature of the stored regeneration fluid.

In an embodiment, the controller is adapted to obtain at least one amplifying fluid configured in use to amplify an interaction to aid detection thereof by a sensor, the controller being configured in use to add the at least one amplifying fluid to one of the plurality of output streams.

In an embodiment, the system further comprises: an amplifying fluid reservoir adapted to temporarily store the at least one amplifying fluid before the controller obtains the at least one amplifying fluid, the amplifying fluid reservoir being configured in use to control the temperature of the stored at least one amplifying fluid.

In an embodiment, the controller is adapted to obtain a buffering fluid configured in use to maintain a predetermined pH in an output stream to which it is added, the controller being configured in use to add the buffering fluid to one of the plurality of output streams.

In an embodiment, one of the plurality of sensors is housed within a sensor cell having an adjustable volume for receiving a portion of one of the plurality of output streams, said one sensor being operable to detect the interaction using the portion of said one output stream received in the sensor cell.

In an embodiment, the controller comprises a pumping device operable to adjust the flow of said one output stream through the sensor cell.

In an embodiment, the pumping device is configured in use to provide an intermittent flow of said one output stream through the sensor cell.

In an embodiment, the controller is configured in use to provide one of the plurality of output streams to the detector at a different time to another of the plurality of output streams.

In an embodiment, the system further comprises: a sampler adapted to receive the fluid from a fluid source and comprising a storage unit for temporarily storing the fluid, the sampler being configured in use to form the one or more sample streams from the fluid stored in the storage unit.

In an embodiment, the system further comprises: a data acquirer adapted to receive electronic data from one of the plurality of sensors, wherein the electronic data indicates detection of the analyte corresponding to said one sensor.

In an embodiment, the data acquirer is operable to receive electronic data from said one sensor only when the pumping device flows said one output stream through the sensor cell.

In an embodiment, said one sensor is additionally operable to detect background noise and include in the electronic data an indication of the background noise detected at said one sensor, and wherein the data acquirer is configured in use to use the indication of the background noise to cancel background noise in the electronic data.

In an embodiment, the system further comprises: a notifier adapted to receive the electronic data from the data acquirer, wherein the notifier is configured in use to generate a notification in dependence on the received electronic data.

In an embodiment, the corresponding detecting agent of one of the plurality of sensors is different to the corresponding detecting agent of another of the plurality of sensors, and wherein the interaction relating to said one sensor indicates the presence of a different corresponding analyte to the interaction relating to said other sensor.

In an embodiment, one of the plurality of sensors is operable to detect a level of interaction using the corresponding detecting agent, wherein the level of interaction indicates an amount of the corresponding analyte which is present.

In an embodiment, the interaction comprises binding together of the corresponding detecting agent and the corresponding analyte, and wherein at least one sensor detects the interaction by detecting a physical change caused by the binding operation.

In an embodiment, the corresponding detecting agent and the corresponding analyte bind together and the interaction comprises unbinding of the corresponding analyte and at least part of the corresponding detecting agent, and wherein at least one sensor detects the interaction by detecting a physical change caused by the unbinding operation.

In an embodiment, the interaction comprises at least one chemical reaction, and wherein at least one sensor detects the interaction by detecting a physical change caused by the at least one chemical reaction.

In an embodiment, the physical change is at least one of the following group: a change in mass, a change in electrical resistivity, a change in electrical conductivity, a change in refractive index, a change in electric current, a change in electric potential.

In an embodiment, the physical change occurs to the at least one sensor.

In an embodiment, the detecting agent is one of the following group: an antibody, a protein, an oligonucleotide, a polymer, an inorganic material, an organic substance.

In an embodiment, one of the plurality of sensors is at least one of the following group: a piezoelectric sensor, an electrochemical sensor, an electrochemical quartz crystal microbalance sensor, a surface plasmon resonance sensor, an oxidation reduction potential sensor.

Various embodiments provide a method for detecting one or more analytes in a fluid, the method comprising: obtaining one or more sample streams of the fluid, forming a plurality of output streams, each output stream comprising at least part of one of the one or more sample streams, detecting one or more interactions using the plurality of output streams and a plurality of sensors, wherein each sensor is operable to detect an interaction between a corresponding detecting agent and a corresponding analyte, and determining if the fluid contains one or more said analytes based on the one or more detected interactions.

In an embodiment, the method further comprises: forming the corresponding detecting agent of one of the plurality of sensors on said one sensor.

In an embodiment, the interaction detected by said one sensor comprises binding of the corresponding analyte to the corresponding detecting agent, and the method further comprises removing the bound corresponding analyte from said one sensor by exposing said one sensor to a flushing fluid configured in use to remove a bound analyte.

In an embodiment, the method further comprises: forming a corresponding detecting agent of one of the plurality of sensors by exposing said one sensor to a regeneration fluid.

In an embodiment, the regeneration fluid comprises the corresponding detecting agent and exposure of said one sensor to the regeneration fluid transfers the corresponding detecting agent to said one sensor.

In an embodiment, the method further comprises: temporarily storing the regeneration fluid before exposing said one sensor to the regeneration fluid; and controlling the temperature of the regeneration fluid whilst it is being temporarily stored.

In an embodiment, the method further comprises: adding at least one amplifying fluid to one of the plurality of output streams, the at least one amplifying fluid being configured in use to amplify an interaction to aid detection thereof by a sensor.

In an embodiment, the method further comprises: temporarily storing the at least one amplifying fluid before adding the at least one amplifying fluid to said one output stream; and controlling the temperature of the at least one amplifying fluid whilst it is being temporarily stored.

In an embodiment, the method further comprises: adding a buffering fluid to one of the plurality of output streams, the buffering fluid being configured in use to maintain a predetermined pH in said one output stream.

In an embodiment, the method further comprises: adjusting a flow of at least one output stream through at least one of the plurality of sensors.

In an embodiment, the method further comprises: adjusting the flow to be intermittent.

In an embodiment, said step of detecting one or more interactions comprises using one of the plurality of output streams at a different time to another of the plurality of output streams.

In an embodiment, the method further comprises: obtaining the fluid from a fluid source; and temporarily storing the obtained fluid before forming the one or more sample streams of the fluid from the temporarily stored fluid.

In an embodiment, the method further comprises: receiving electronic data from one of the plurality of sensors, wherein the electronic data indicates detection of the analyte corresponding to said one sensor.

In an embodiment, electronic data is received from said one sensor only when said one output stream flows through said one sensor.

In an embodiment, said one sensor is additionally operable to detect background noise and include in the electronic data an indication of the background noise detected at said one sensor; and the method further comprises cancelling background noise in the electronic data using the indication of the background noise.

In an embodiment, the method further comprises: generating a notification in dependence on the received electronic data.

In an embodiment, the corresponding detecting agent of one of the plurality of sensors is different to the corresponding detecting agent of another of the plurality of sensors, and wherein the interaction relating to said one sensor indicates the presence of a different corresponding analyte to the interaction relating to said other sensor.

In an embodiment, the method further comprises: detecting a level of interaction using one of the plurality of sensors, wherein the level of interaction indicates an amount of the corresponding analyte which is present.

In an embodiment, the interaction comprises binding together of the corresponding detecting agent and the corresponding analyte; and, the method further comprises detecting the interaction by detecting a physical change caused by the binding operation.

In an embodiment, the corresponding detecting agent and the corresponding analyte bind together and the interaction comprises unbinding of the corresponding analyte and at least part of the corresponding detecting agent; and, the method further comprises detecting the interaction by detecting a physical change caused by the unbinding operation.

In an embodiment, the interaction comprises at least one chemical reaction; and, the method further comprises detecting the interaction by detecting a physical change caused by the at least one chemical reaction

In an embodiment, the physical change is at least one of the following group: a change in mass, a change in electrical resistivity, a change in electrical conductivity, a change in refractive index, a change in electric current, a change in electric potential.

In an embodiment, the physical change occurs to the corresponding sensor.

In an embodiment, the detecting agent is one of the following group: an antibody, a protein, an oligonucleotide, a polymer, an inorganic material, an organic substance.

In an embodiment, one of the plurality of sensors is at least one of the following group: a piezoelectric sensor, an electrochemical sensor, an electrochemical quartz crystal microbalance sensor, a surface plasmon resonance sensor, an oxidation reduction potential sensor.

### BRIEF DESCRIPTION OF THE DRAWINGS

Embodiments of the invention will be better understood and readily apparent to one of ordinary skill in the art from the following written description, by way of example only, and in conjunction with the drawings, wherein like reference signs relate to like components, in which:

FIG. 1 illustrates a system for detecting one or more analytes in a fluid in accordance with an embodiment;

FIG. 2 illustrates a method for detecting one or more analytes in a fluid in accordance with an embodiment;

FIG. 3 illustrates a system for detecting one or more analytes in a fluid in accordance with an embodiment;

FIG. 4 illustrates a sampler in accordance with an embodiment;

FIG. 5 illustrates a system for detecting one or more analytes in a fluid in accordance with an embodiment;

FIG. 6 illustrates a sensor cell in accordance with an embodiment;

FIGS. 7A and 7B illustrate a sensor in accordance with an embodiment,

FIG. 7A showing a front view and FIG. 7B showing a side view;

FIG. 8 illustrates a computer system in accordance with an embodiment;

FIG. 9 illustrates a method for detecting one or more analytes in a fluid in accordance with an embodiment;

FIG. 10 illustrates a method for detecting one or more analytes in a fluid in accordance with an embodiment;

FIG. 11 illustrates a method for detecting one or more analytes in a fluid in accordance with an embodiment; and

FIG. 12 illustrates sensor coating recipes according to various embodiments.

### DETAILED DESCRIPTION

Various embodiments relate to a system and method for detecting one or more analytes in a fluid.

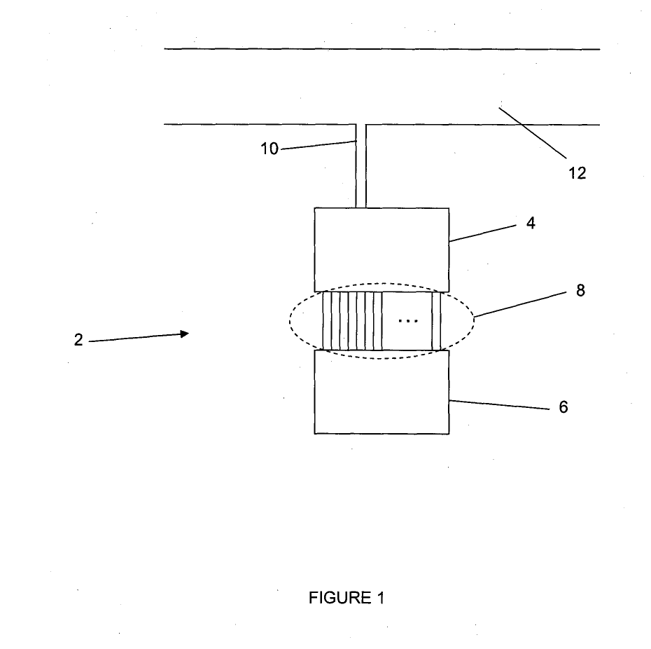

FIG. 1 illustrates a system 2 for detecting one or more analytes in a fluid, in accordance with an embodiment. In an embodiment, the system 2 includes a controller 4 in communication with a sensing device or detector 6. In an embodiment, the controller 4 is in communication with the detector 6 via a plurality of output streams 8 of fluid. In an embodiment, each output stream 8 may be contained within a tube which is in fluid communication with both the controller 4 and the detector 6.

In an embodiment, the controller 4 is also in communication with at least one sample stream 10 of fluid. In an embodiment, the fluid of the sample stream 10 is obtained from a fluid source 12. In an embodiment, the sample stream 10 may be contained within a tube which is in fluid communication with both the fluid source 12 and the controller 4. However, in another embodiment, no fluid source 12 is present and the sample stream 10 is obtained directly from a tube in fluid communication with the controller 4.

In an embodiment, the controller 4 is configured in use to obtain at least one sample stream 10 of the fluid. For example, the controller 4 may obtain the sample stream 10 by virtue of a direct or indirect coupling to a tube containing the sample stream. In an embodiment, the controller 4 may obtain the sample stream 10 via a pumping device (not shown), i.e. the controller 4 may pump the sample stream 10 to the controller 4, for example, from a fluid source 12.

In an embodiment, the controller 4 is configured in use to form the plurality of output streams 8 using at least one sample stream 10. In an embodiment, each output stream 8 includes at least part of one sample stream 10. In an embodiment, the controller 4 may form the plurality of output streams 8 by combining and/or splitting one or more sample streams 10 using a pumping device (not shown) and a switching device (not shown). In an embodiment, the switching device may include a network of tubes and valves which are configured in use to channel at least part of each input sample stream 10 into one or more of the plurality of output streams 8.

In an embodiment, the detector 6 is adapted to receive from the controller 4 the plurality of output streams 8. For example, the controller 4 may include a pumping device configured in use to pump each output stream 8 to the detector 6. In an embodiment, the detector 6 includes a plurality of sensors. In an embodiment, the plurality of sensors are arranged to form a sensor array or a sensor bank. In an embodiment, the detector 6 includes a different sensor for each output stream 8 provided by the controller 4. In an embodiment, the controller 4 is configured to provide a plurality of output streams 8 to the plurality of sensors of the detector 6, such that the system 2 provides a multiple channel sensor system 2 for detecting one or more analytes in a fluid. Specifically, each channel may be considered as one fluid connection between an output stream 8 and a sensor.

In an embodiment, each sensor is operable to detect an interaction using a corresponding detecting agent in the presence of a corresponding analyte. In an embodiment, the interaction may be a physical interaction or a chemical interaction. In an embodiment, the sensor is a biosensor configured to detect a change in a physical property caused by the interaction. In an embodiment, the physical property is a physical property of the sensor itself. In an embodiment, the biosensor is a piezoelectric biosensor which detects a change in vibration frequency caused by the interaction. In an embodiment, the biosensor is a surface plasmon resonance biosensor which detects a change in optical properties caused by the interaction. In an embodiment, the biosensor is an electrochemical biosensor which detects a change in electrical properties caused by the interaction.

In an embodiment, the analyte corresponds with the detecting agent and visa versa. In other words, the detecting agent and analyte form an associated set, wherein each member of the set interacts with the other member when they are put together. In an embodiment, the detecting agent corresponds with the sensor. In an embodiment, one or more of the sensors are associated with, i.e. correspond with, a different detecting agent. In another embodiment, one or more sensors correspond with the same detecting agent. In an embodiment, a coating which is applied to an external surface of a sensor includes the detecting agent corresponding to that sensor. In another embodiment, the controller 4 provides to the sensor a fluid which includes the detecting agent corresponding to that sensor.

In an embodiment, the detector 6 is configured in use to detect one or more interactions using the plurality of output streams 8 to determine if one or more analytes are present in the fluid from which the sample streams 10 were taken. Specifically, each sensor is operable to detect an interaction caused when a detecting agent corresponding to the sensor is combined with an analyte corresponding to the detecting agent. Accordingly, if the sensor includes an external coating containing the detecting agent, and the sensor is exposed to an output stream 8 containing the analyte, an interaction may occur and the sensor may detect the interaction. Therefore, the sensor may be used to detect the presence of the analyte in an output stream 8 to which the sensor is exposed. If the plurality of output streams 8 is provided to the plurality of sensors, multiple analytes may be detected. In an embodiment, each output stream 8 is provided to a different sensor, and each different sensor is configured to detect a different analyte. Therefore, detection of a plurality of different analytes in the output streams 8 may be performed. In an embodiment, each output stream 8 may originate from the same fluid, therefore, detection of one or more analytes in that fluid may be performed.

In an embodiment, one or more output streams 8 may originate from a different fluid source 12 to one or more other output streams 8, i.e. they may contain sample streams 10 from different fluid sources 12. In an embodiment, one or more of the sensors may detect the same analyte. In an embodiment, one or more of the sensors may detect a different analyte. In an embodiment, a single sensor may be operable to detect multiple different analytes. For example, the sensor may include an external coating comprising two or more detecting agents, wherein two or more of these detecting agents each cause an interaction in the presence of a different analyte.

In an embodiment, the controller 4 is configured to control the time at which each sample stream 10 is obtained. In an embodiment, the controller 4 is configured to control the time at which each output stream 8 is provided to a sensor of the detector 6. Accordingly, the controller 4 may be configured to hold or store fluid during its transition from a sample stream 10 to an output stream 8. Furthermore, in an embodiment, the controller 4 may be configured in use to provide some or all of the output streams 8 to the detector 6 for analyte detection at substantially the same time. In this way, the system 2 may be configured in use to simultaneously detect the presence of different analytes in the fluid. In an embodiment, the controller 4 may be configured in use to provide some or all of the output streams 8 to the detector 6 for analyte detection at different times, such as, for example, in an ordered sequence. In this way, the system 2 may be configured in use to periodically detect the presence of a single analyte in a fluid. Stated differently, the controller 4 may be configured in use to implement a queuing system 2. Accordingly, different samples and, therefore, different output streams 8, may be held in the queue such that they are only examined for analytes once it is their turn.

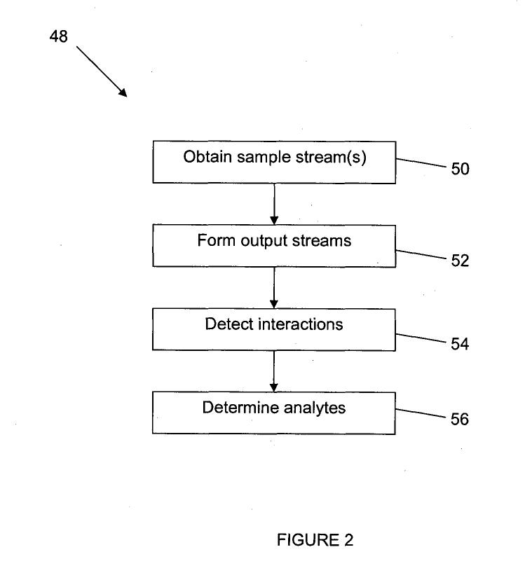

FIG. 2 provides a flow diagram 48 of the above-described operation in accordance with various embodiments.

At 50, one or more sample streams 10 of a fluid under test are obtained. In an embodiment, the or each sample stream 10 is pumped from a fluid source. In an embodiment, the fluid source relating to one or more sample streams 10 may be different to the fluid source relating to one or more other sample streams 10.

At 52, the or each sample stream 10 is used to form a plurality of output streams 8, wherein each output stream includes at least part of an obtained sample stream 10. In an embodiment, the plurality of output streams 8 is formed by combining and/or splitting-up obtained sample streams 10.

At 54, one or more interactions are detected using the plurality of output streams 8 and a plurality of sensors. In an embodiment, each sensor is exposed to a different one of the plurality of output streams 8. In an embodiment, each sensor is operable to detect an interaction using a corresponding detecting agent in the presence of a corresponding analyte. Accordingly, detection of an interaction by a sensor may indicate the presence of a specific analyte associated with the sensor. In an embodiment, an external coating of the sensor includes the detecting agent. In another embodiment, the controller 4 provides the detecting agent to the sensor in a fluid.

At 56, it is determined whether or not the fluid contains one or more analytes based on which sensors detected interactions. In an embodiment, the specific analytes contained within the fluid may be identified based on which specific sensors detected an interaction.

In the above-described embodiments, analyte detection may be indicated by any suitable indicator. For example, the detector 6 may provide a light and/or a sound to indicate that one or more particular analytes are present. In an embodiment, the detector 6 may include a display screen; and the detector 6 may present the detection results on the display screen.

According to the above-described embodiments, it is possible to detect the presence of one or more analytes in a fluid. In an embodiment, the fluid is water and each detected analyte indicates the presence of an impurity. Accordingly, an advantage of the above-described embodiments is that water quality can be determined and monitored. Another advantage is that many different analytes may be detected simultaneously or sequentially. Therefore, many different fluid parameters may be detected. In an embodiment, it is an advantage that many different water parameters and water impurities can be detected and monitored.

An advantage of the above-described embodiments is that multiple analytes may be detected simultaneously since one or more of the sensors may be exposed to an output stream at substantially the same time as one or more other sensors. It is also an advantage that different sensors may be exposed to an output stream 8 at different times. Accordingly, periodic monitoring of the presence of one or more analytes may be achieved.

It is an advantage of the above-described embodiments that continuous monitoring may be achieved. Specifically, a sequence of sample streams 10 may be converted into a corresponding sequence of output streams 8 and exposed to a detector 6 sensor in the time order in which they were generated. For example, a first output stream comprising a first sample stream 10 may be exposed to a first sensor for detection at a first time. However, detection may require ten time units to perform. Accordingly, during the subsequent nine time units, second to tenth corresponding operations may be performed in sequence. It is then possible to provide a detection result every time unit following the tenth time unit, in spite of the fact that it takes ten time units to generate a single detection result. This process may be looped in order to continue to provide one detection result every time unit. Accordingly, continuous and real-time monitoring may be achieved.

The following describes a system comprising a multiple channel piezoelectric sensor array in accordance with various embodiments.

Analysis of water quality parameters can be performed off-line using wet chemistry methods. These methods require costly and bulky instruments, long operation time, skilled personnel and the usage of toxic chemicals. For some parameters, the analysis time required may be hours or days. Some of these methods have been automated and utilized in-line with a water system. However, the tedious procedures which they necessarily include hinder their applications for online, real-time and continuous monitoring of water quality. Moreover, the amount of toxic chemicals introduced into the water system increases as the demand for water quality analysis increases. The usage and disposal of these chemicals can become a significant public concern.

Also, optical spectral methods can be used to monitor water quality parameters, such as, nitrates and nitrites, chemical oxygen demand (COD), biochemical oxygen demand (BOD) and total organic carbon (TOC) containing substances absorbing light in Ultraviolet (UV) to visible spectra range. However, these methods do not respond to compounds without unsaturated bonds, such as aliphatic amines, amino acids and carbohydrates. Also, the accuracy of the results obtained from these methods depends largely on the composition of the water samples used and the localized calibration. Accordingly, the accuracy may vary significantly between applications, for example, in a range from 60% to 90%. Furthermore, only a few fundamental water quality parameters such as pH, turbidity and conductivity can be reliably analyzed online using these simple physical methods.

In an embodiment, at least one of the sensors of the detector is a piezoelectric sensor. In an embodiment, the piezoelectric sensor includes quartz crystals. Piezoelectric sensors are advantageous because they provide good sensitivity at a relatively low-cost. In an embodiment, the piezoelectric sensor is configured such that when its surface interacts with a target species (i.e. an analyte) a change is produced in the sensor's physical properties, such as, for example, vibration frequency, surface impedance and surface acoustic wave. For example, the frequency change (Δf) due to mass change (Δm) can be expressed by the following Sauerbrey equation formulae (1):

$$Δ f = \frac{- 2 Δ {mf}_{0}^{2}}{A \sqrt{{ρ}_{q} {μ}_{q}}} = - \frac{2 {f}_{0}^{2}}{A \sqrt{{ρ}_{q} {μ}_{q}}} Δ m \qquad ( 1 )$$

wherein f₀ is the resonant frequency (Hz) of the sensor; A is the piezoelectrically active crystal area (cm²); ρ_{q} is the density of quartz (2.648 g/cm³); and, μ_{q} is the shear modulus of quartz for AT-cut crystal (2.947×1011 g/cm·s²).

In an embodiment, an external surface of the piezoelectric sensor is coated with one or more receptors (i.e. detecting agents), such as, for example, antibodies and polymers that can interact with a specific type of group of targets (i.e. analytes).

In an embodiment, the piezoelectric sensor may be configured to additionally take electrochemical measurements. In an embodiment, the sensor may be an electrochemical quartz crystal microbalance (EQCM) sensor. However, in an embodiment, differently from conventional EQCM methods, the EQCM sensor may be used to measure oxidation reduction potential (ORP) which is an indicator for general water quality parameters such as nitrate, COD, BOD, and TOC.

The following provides a detailed description of a system for detecting one or more analytes in a fluid containing the above-mentioned sensor in accordance with an embodiment.

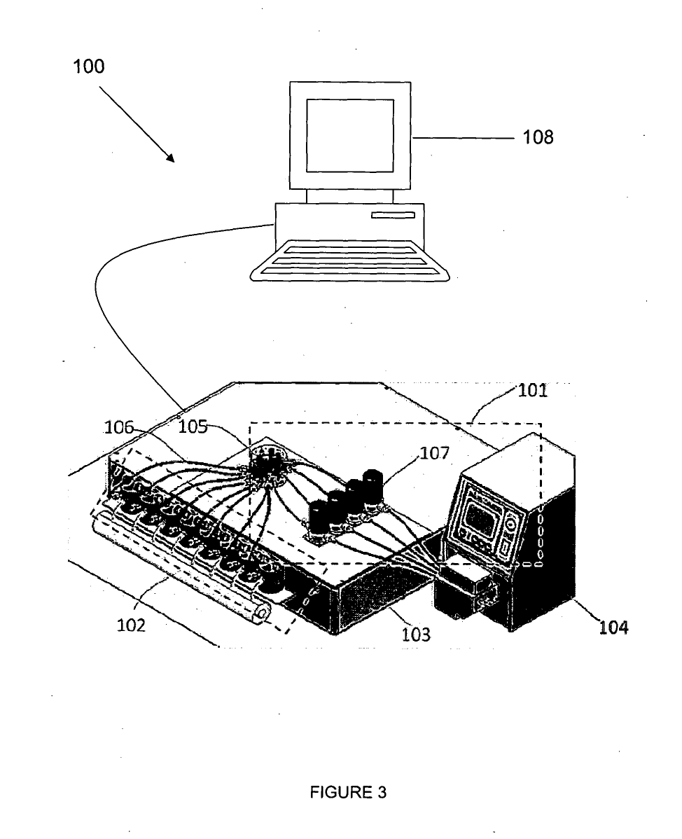

FIG. 3 shows a system 100 for online water quality monitoring in accordance with an embodiment. In an embodiment, the system 100 includes a sampler (not shown) to introduce water from a water source or piping system. In an embodiment, the system 100 includes a controller 101 to distribute fluid sample and other fluids to detector 102 sensors in an appropriate order. In an embodiment, the system 100 includes an array of sensor cells with drainage (i.e. a detector) 102. In an embodiment, each sensor cell is equipped with piezoelectric sensors that sense species (i.e. analytes) contributing to a range of water quality parameters. In an embodiment, the system 100 includes a data acquisition module (i.e. a data acquirer) 103 with a microprocessor that converts physical signals to digital data before data processing. In an embodiment, the system includes a reporting module (i.e. a notifier) 108 to display real-time results and send notifications.

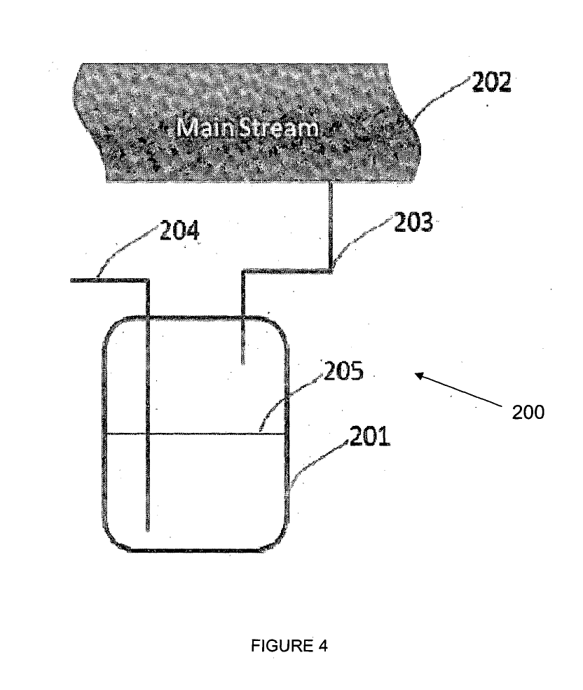

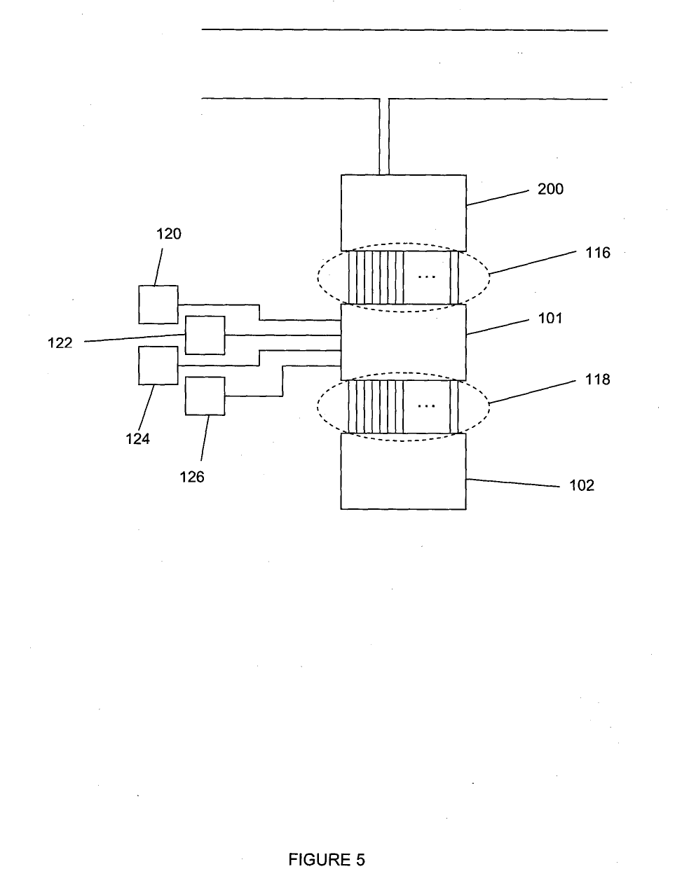

FIGS. 4 and 5 illustrate in more detail a sampler (i.e. a sampling portion) 200 in accordance with an embodiment. In an embodiment, the sampler 200 includes a sample collection container (i.e. a storage unit) 201. In an embodiment, the sampler 200 includes an inlet 203 which links the sample collection container 201 to a main water stream 202. In an embodiment, the sampler 200 includes an outlet 204 which links the sample collection container 201 to the controller 101. In an embodiment, the outlet 204 is submerged under a fluid level 205 and near a bottom of the sample collection container 201. Accordingly, the sampler 200 may include tubes and valves, such as inlet 203 and outlet 204, which connect the sample collection container 201 on the one side to the main piping stream 202, and on the other side to the controller 101 (FIG. 3). In an embodiment, the sampler 200 is configured to provide one or more sample streams 116 to the controller 101. In an embodiment, the collection container 201 is configured in use to buffer the water sample stream from the water source. Stated differently, the container 201 temporarily stores water from the water source. This operation of the sampler 200 may be controlled via one or more valves. Accordingly, the sampler 200 may act as a buffer between the main water supply and the controller 101. Therefore, the sampler 200 may protect the controller 101 from damage caused by the pressure and flow rate of fluid in the main water supply.

As seen more particularly in FIG. 3, in an embodiment, the controller 101 includes a pumping device 104 and a switching device 105. In an embodiment the pumping device 104 may include one or more pumps. In an embodiment, the or each pump is configured in use to provide an adjustable flow rate. In an embodiment, the pumping device 104 is configured to control the fluid streams flowing through tubes 106.

As seen more particularly in FIG. 5, in an embodiment, in addition to sample streams 116, the controller 101 is in fluid communication with containers (or reservoirs) of buffering fluid 120, regeneration fluid 122, amplifying fluid 124 and/or flushing fluid 126. In an embodiment, the tubes 106 are connected at one end to containers of samples 116, flushing fluids 126, buffering fluids 120, amplifying fluids 124 and/or regenerating fluids 122. In an embodiment, the tubes 106 are connected at the other end to inputs of the switching device 105. In an embodiment, the outputs of the switching device 105 provide, or are connected to, the outputs of the controller 101. In an embodiment, the outputs of the controller 101 are connected to the inputs of the detector 102. In an embodiment, the inputs of the detector 102 are connected to the plurality of sensors.

As seen more particularly in FIG. 3, in an embodiment, the direction of fluid flow in the tubes 106 and/or the switching device 105 is controlled by a series of valves 107. In an embodiment, the valves 107 are also configured in use to permit or prevent fluid flow in the tubes 106 and/or the switching device 105. In an embodiment, the values 107 and tubes 106 provide contamination control and fluidic holding. In an embodiment, the controller 101 includes the tubes 106 and the valves 107.

In an embodiment, the buffering fluid 120 is configured in use to maintain a predetermined pH in the fluid to which it is added. In an embodiment, the buffering fluid 120 includes two parts, an acidic part and an alkaline part, wherein the two parts are mixed together in appropriate proportions by the controller 101 to provide a mixture having a predetermined pH. For example, it may be the case that an interaction between a detecting agent and an analyte may occur best, or may provide a more easily detectable interaction, when the fluid in which the interaction occurs is at a particular pH. Accordingly, the controller 101 may be configured in use to obtain buffering fluid 120 and add it to one or more of output streams 118. Accordingly, interaction detection and, therefore, analyte detection may be more reliable.

In an embodiment, the buffering fluid 120 is contained within a reservoir which is in fluid communication with the controller 101. In an embodiment, the controller 101 may be configured to provide one or more different buffering fluids 120 to an output stream 118. In an embodiment, each different buffing fluid 120 may be stored in a different reservoir or a different partition or portion of the same reservoir.

In an embodiment, a sensor includes an external coating having the corresponding detecting agent. Accordingly, when an analyte corresponding to the detecting agent is present, the analyte binds with the detecting agent. This binding action provides the interaction which may be detected by a sensor to indicate that the analyte is present. However, following detection, the analyte bound to the external surface of the sensor may be removed, for example, to permit subsequent separate sensing operations. Accordingly, in an embodiment, the controller 101 is adapted to obtain a flushing fluid 126 which is configured in use to remove a bound analyte. In an embodiment, the controller 101 is configured in use to provide the flushing fluid 126 to one or more sensors of the detector 102 to remove bound analytes. Accordingly, the controller 101 may prepare the sensor for subsequent separate sensing operations. In an embodiment, the flushing fluid 126 may be the same as the buffering fluid 120.

In an embodiment, the flushing fluid 126 is contained within a reservoir which is in fluid communication with the controller 101. In an embodiment, the controller 101 may be configured to provide one or more different flushing fluids 126 to one or more sensors of the array. In an embodiment, each different flushing fluid 126 may be stored in a different reservoir or a different partition or portion of the same reservoir.

In an embodiment, a detecting agent is provided to a sensor via a regeneration fluid 122. For example, the regeneration fluid 122 may react with the sensor surface to form the detecting agent on the sensor surface. Alternatively, the regeneration fluid 122 may include the detecting agent such that contacting the sensor surface with the regeneration fluid 122 transfers the detecting agent to the sensor surface. In any case, in an embodiment, the controller 101 is configured in use to obtain a regeneration fluid 122 and provide the regeneration fluid 122 to one or more sensors of the detector 102. In this way, the controller 101 may prepare the one or more sensors for interaction detection and, therefore, analyte detection.

In an embodiment, the regeneration fluid 122 is contained within a reservoir which is in fluid communication with the controller 101. In an embodiment, the controller 101 may be configured to provide one or more different regeneration fluids 122 to one or more sensors of the array. In an embodiment, each different regeneration fluid 122 may be stored in a different reservoir or a different partition or portion of the same reservoir.

In an embodiment, the amplifying fluid 124 is configured in use to amplify an interaction which occurs in a fluid to which it is added, thereby aiding detection of the interaction. For example, it may be the case that an interaction between a detecting agent and an analyte does not produce a significant enough interaction to be reliably detected. In an embodiment, the interaction may be amplified by the analyte binding to the amplifying fluid. Accordingly, the controller 101 may be configured in use to obtain amplifying fluid 124 and add it to one or more of output streams 118 in advance of sending that output stream 118 to a sensor for analyte detection. Accordingly, interaction detection and, therefore, analyte detection may be improved.

In an embodiment, the amplifying fluid 124 is contained within a reservoir which is in fluid communication with the controller 101. In an embodiment, the controller 101 may be configured to provide one or more different amplifying fluids 124 to an output stream 118. In an embodiment, each different amplifying fluid 124 may be stored in a different reservoir or a different partition or portion of the same reservoir.

Various embodiments include a reservoir for temporarily storing a fluid, such as, for example, a buffering fluid 120, a flushing fluid 126, a regenerating fluid 122, and/or an amplifying fluid 124. In an embodiment, such fluids are temporarily stored before the controller 101 obtains the fluid, for example, for provision to a sensor or for addition to an output stream. In an embodiment, the reservoir is configured in use to control the temperature of the fluid stored therein. In an embodiment, the reservoir is configured in use to control the temperature of the fluid leaving the reservoir. For example, the reservoir may include a heat sink to remove heat from the fluid to lower the fluid temperature. Additionally or alternatively, the reservoir may include a heat exchanger to add heat to the fluid to raise the fluid temperature. Accordingly, it is an advantage that where a fluid performs optimally at a particular temperature, or within a particular temperature range, the system may be configured in use to ensure that the fluid is maintained substantially at that particular temperature, or within that particular temperature range, when it is being used. Additionally, it is an advantage that where a fluid lasts longer when stored at a particular temperature, or within a particular temperature range, the system may be configured in use to ensure that the fluid is maintained substantially at that particular temperature, or within that particular temperature range, when it is being stored.

In an embodiment, a fluid sample may also be temporarily stored in a reservoir before use by the controller 101. Accordingly, it may be possible to set the temperature of the output stream by controlling the temperature of the fluid sample, rather than, or in addition to, controlling the temperature of the buffering fluid 120, the flushing fluid 126, the regenerating fluid 122, and/or the amplifying fluid 124.

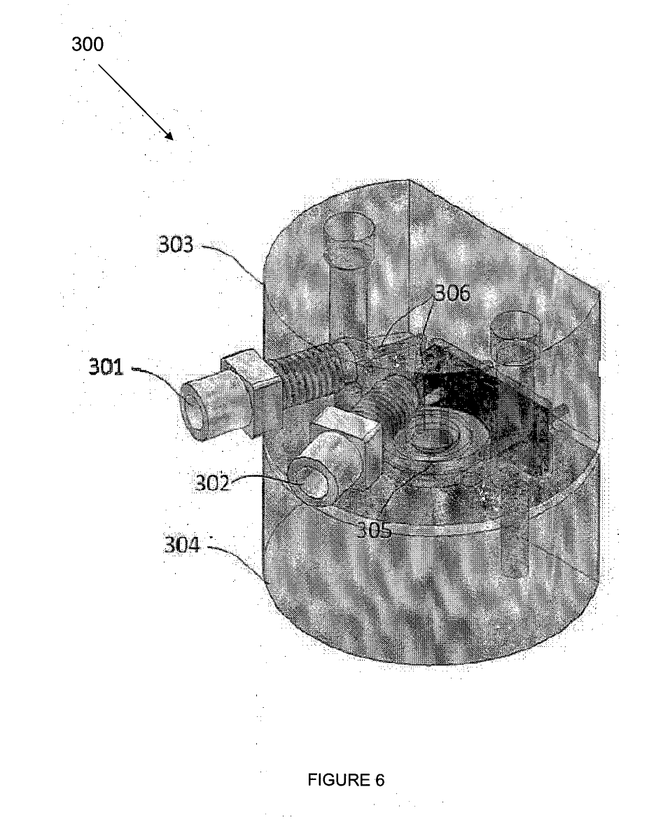

FIG. 6 illustrates a sensor cell 300 in accordance with an embodiment. In an embodiment, the sensor cell 300 has an inlet 301 and an outlet 302, both of which are equipped with a connector for a tube, such as, a tube 106 of the controller 101. In an embodiment, the sensor cell 300 includes an upper portion 303 and a lower portion 304 which are arranged together to define a sensor chamber 305. In an embodiment, the sensor chamber 305 is configured in use to house one or more sensors of the detector 102. In an embodiment, the sensor chamber 305 is formed in between the upper and lower portions by pressing the two portions together with screws, nuts and/or any other mechanical or chemical fasteners. In an embodiment, a volume of the sensor chamber 305 is adjustable, and may range from microlitres to litres. In an embodiment, the sensor chamber 305 is sealed water tight at the joint between the upper and lower portions with one or more rubber O-rings (not shown). In an embodiment, the chamber 305 is linked to the inlet 301 and the outlet 302 via two internal flowing channels 306. In an embodiment, the widths of the channels 306 are identical. In an embodiment, the widths of the channels 306 are not more than about 1.0 mm.

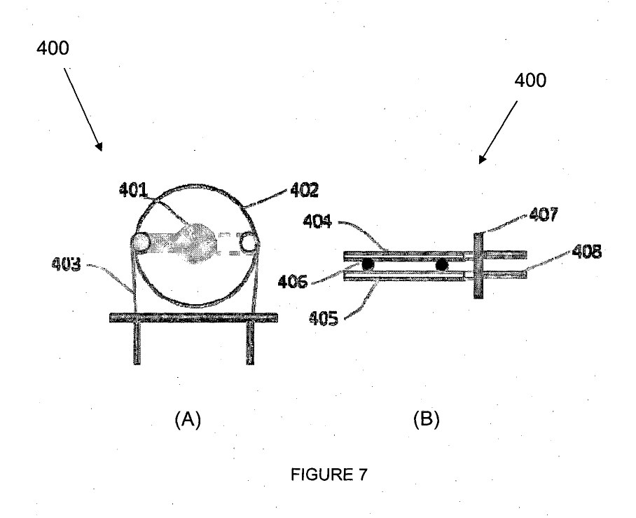

FIG. 7 illustrates a sensor 400 according to various embodiments. In an embodiment, the sensor 400 is a dual-crystal piezoelectric sensor for real-time noise elimination.

In an embodiment, a dual-crystal piezoelectric sensor 400 including two quartz crystals 401 is inserted into the above-mentioned sensor chamber 305 (FIG. 6). In an embodiment, gold 402 is coated on both sides of the crystals 401 and connected to metal electrode wires 403. In an embodiment, a chromium or titanium interlayer is coated between the gold 402 and the crystal 401 to improve affinity of the gold onto the crystals 401. In an embodiment, the thickness of the interlayer is about 1.0 nm. In an embodiment, the thickness of the gold layer is about 100.0 nm. In an embodiment, considering the two crystals 401, one crystal 404 is for detecting an analyte, while the other crystal 405 is for background noise referencing. In an embodiment, both crystals are mounted in a parallel formation and in close proximity to each other. In an embodiment, each crystal is physically separated from the other crystal by an isolating or screening spacer 406. In an embodiment, each crystal is electrically isolated from the other crystal by an isolation pad 407.

In an embodiment, the dual-crystal piezoelectric sensor 400 has four metal electrode pins 408 connecting to two electrodes of the two sensor crystals 401. In an embodiment, the two sensor crystals 401 are linked in parallel in a circuit by plugging the four pins 408 in connectors which are linked to a circuit board equipped with an oscillation generator, a frequency counter and a microprocessor. In an embodiment, the two sensor crystals 401 are linked to the circuit board separately. In an embodiment, the circuit board contains eight channels of data acquisition, each of which contains two sets of identical circuits connecting to two sensor crystals 401 of one dual-crystal piezoelectric sensor 400. In an embodiment, data acquired by the referencing crystal 405 is used to cancel off background noise and interference in the data from the sensing crystal 404.

In an embodiment, the dual crystal piezoelectric sensor 400 includes a built-in ORP electrode for complementary COD/BOD/TOC analysis. In an embodiment, as in an EQCM sensor, the gold electrode on the quartz crystal surface can be used as a working electrode of an ORP electrode. In an embodiment, a metal wire is placed in the sample chamber 301 of a sensor cell 300 as a counter electrode. In an embodiment, suitable electrode metals are gold, platinum and nickel. In an embodiment, an optional reference electrode can also be used to improve the accuracy of the ORP measurement.

In an embodiment, the system 100 may be configured to perform data processing and reporting. As seen more particularly on FIG. 3, the system may include the data acquirer 103 for collecting electronic data from the sensors, for example, through one or more analog-to-digital convertors. In an embodiment, electronic data may be processed using mathematical methods, such as, the Fourier Transform, for example, to eliminate noise in the electronic data. In an embodiment, concentration of pollutants in water and water quality parameters are calculated using statistical methods, such as, for example, principal component analysis and partial least squares regression.

In an embodiment, it may be the case that analyte detection is best when performed at a particular temperature, or within a particular temperature range. For example, this temperature or temperature range may be dependent on the precise composition of the output stream. However, it may not have been possible to bring that output stream to the particular temperature or within the particular temperature range. Accordingly, it may be possible to measure the actual temperature of the output stream and adjust the electronic data received from the detector 102 to compensate for the difference between the actual temperature and the optimum temperature. Accordingly, the data acquirer 103 may be configured to perform a temperature compensation algorithm.

In an embodiment, the sensors 400 are operable to detect a level of interaction, wherein the level of interaction indicates an amount of the corresponding analyte which is present. In an embodiment, the level of interaction corresponds to the quantity of interactions detected by the sensor. An advantage of this operation is that the amount of an analyte present in a sample may be detected, rather than simply detecting the presence of the analyte. For example, the fluid may be water and the analyte may relate to a particular component of the water. In an embodiment, the presence of only a small quantity of the analyte may not make the water unfit for human consumption; however, the presence of a large quantity may make the water unfit for human consumption. Therefore, it is an advantage that the system according to various embodiments may detect an amount of analyte present.

As seen more particularly on FIG. 3, the system 100 may include the notifier 108 configured in use to display real-time results on a display screen. In an embodiment, the data acquirer 103 may be configured to calculate one or more values relating to a quantity (i.e. level) of analyte detected by one or more sensors. Accordingly, one or more thresholds may be established, wherein each threshold relates to a level of a particular analyte detected. Accordingly, the notifier 108 may send notifications to prescribed recipients in dependence on the detected amount of a particular analyte compared to the value of the corresponding threshold. Accordingly, in an embodiment, notifications may be sent when water quality drops below certain thresholds. In an embodiment, the notifications can include warnings, alarms, and process control commands, or any combination thereof depending on the detected level. In an embodiment, the notifier 108 is configured in use to send notifications as, for example, an email via the internet and a wifi network and/or a short message service (SMS) message via a GSM or 3G cellular network.

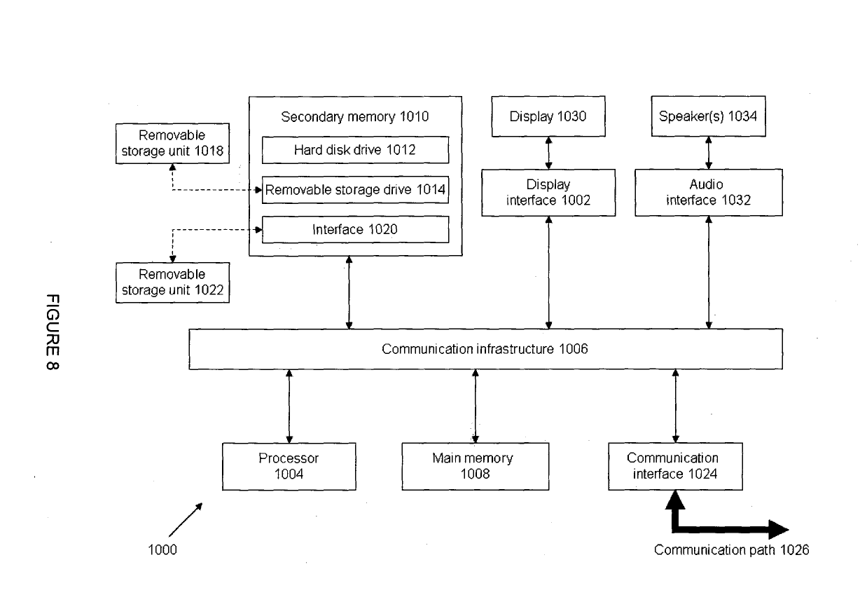

In an embodiment, the role of the data acquirer 103 and/or the notifier 108 may be performed by a computing device. FIG. 8 depicts an example computing device 1000. The following description of computing device 1000 is provided by way of example only and is not intended to be limiting.

As shown in FIG. 8, example computing device 1000 includes a processor 1004 for executing software routines. Although a single processor is shown for the sake of clarity, computing device 1000 may also include a multi-processor system. Processor 1004 is connected to a communication infrastructure 1006 for communication with other components of computing device 1000. Communication infrastructure 1006 may include, for example, a communications bus, cross-bar, or network.

Computing device 1000 further includes a main memory 1008, such as a random access memory (RAM), and a secondary memory 1010. Secondary memory 1010 may include, for example, a hard disk drive 1012 and/or a removable storage drive 1014, which may include a floppy disk drive, a magnetic tape drive, an optical disk drive, or the like. Removable storage drive 1014 reads from and/or writes to a removable storage unit 1018 in a well known manner. Removable storage unit 1018 may include a floppy disk, magnetic tape, optical disk, or the like, which is read by and written to by removable storage drive 1014. As will be appreciated by persons skilled in the relevant art(s), removable storage unit 1018 includes a computer readable storage medium having stored therein computer executable program code instructions and/or data.

In an alternative implementation, secondary memory 1010 may additionally or alternatively include other similar means for allowing computer programs or other instructions to be loaded into computing device 1000. Such means can include, for example, a removable storage unit 1022 and an interface 1020. Examples of a removable storage unit 1022 and interface 1020 include a program cartridge and cartridge interface (such as that found in video game console devices), a removable memory chip (such as an EPROM or PROM) and associated socket, and other removable storage units 1022 and interfaces 1020 which allow software and data to be transferred from the removable storage unit 1022 to computer system 1000.

Computing device 1000 also includes at least one communication interface 1024. Communication interface 1024 allows software and data to be transferred between computing device 1000 and external devices via a communication path 1026. In various embodiments, communication interface 1024 permits data to be transferred between computing device 1000 and a data communication network, such as a public data or private data communication network. Examples of communication interface 1024 can include a modem, a network interface (such as Ethernet card), a communication port, and the like. Software and data transferred via communication interface 1024 are in the form of signals which can be electronic, electromagnetic, optical or other signals capable of being received by communication interface 1024. These signals are provided to the communication interface via communication path 1026. For example, the computing device 1000 may use the communication interface 1024 to communicate with the internet and/or external devices wirelessly via WiFi, 3G/4G, GSM, Bluetooth, Near Field Communication, etc. In an embodiment, the computing device 1000 is configured to communicate with a remote storage volume (not shown) such that data may be stored in and retrieved from the remote storage volume. In an embodiment, the computing device 1000 may communicate with the remote storage volume using the at least one communication interface 1024. In an embodiment, the remote storage volume is a cloud storage system.

As shown in FIG. 8, computing device 1000 further includes a display interface 1002 which performs operations for rendering images to an associated display 1030 and an audio interface 1032 for performing operations for playing audio content via associated speaker(s) 1034.

As used herein, the term “computer program product” may refer, in part, to removable storage unit 1018, removable storage unit 1022, a hard disk installed in hard disk drive 1012, or a carrier wave carrying software over communication path 1026 (wireless link or cable) to communication interface 1024. A computer readable medium can include magnetic media, optical media, or other recordable media, or media that transmits a carrier wave or other signal. These computer program products are devices for providing software to computer system 1000.

Computer programs (also called computer program code) are stored in main memory 1008 and/or secondary memory 1010. Computer programs can also be received via communication interface 1024. Such computer programs, when executed, may enable the computing device 1000 to perform one or more features of the data acquirer and/or notifier disclosed herein. In various embodiments, the computer programs, when executed, enable the processor 1004 to perform features of the data acquirer and/or notifier disclosed herein. Accordingly, such computer programs represent controllers of the computer system 1000.

Software may be stored in a computer program product and loaded into computing device 1000 using removable storage drive 1014, hard disk drive 1012, or interface 1020. Alternatively, the computer program product may be downloaded to computer system 1000 over communications path 1026. The software, when executed by the processor 1004, causes the computing device 1000 to perform functions of the data acquirer 103 and/or notifier 108 disclosed herein.

It is to be understood that the embodiment of FIG. 8 is presented merely by way of example. Therefore, in some embodiments one or more features of the computing device 1000 may be omitted. Also, in some embodiments, one or more features of the computing device 1000 may be combined together. Additionally, in some embodiments, one or more features of the computing device 1000 may be split into one or more component parts.

An advantage of the above-described embodiment is that a multiple channel piezoelectric sensor system is provided for online monitoring of water quality parameters. In an embodiment, the system includes an array of piezoelectric sensors with different types of receptor coatings for detection of multiple water quality parameters covering inorganic, organic and biological species. Stated differently, different sensors may include different coatings of detecting agents, wherein each coating may be capable of detecting a different analyte.

An advantage of the above-described embodiments is that a controller may draw water samples from a water source or piping system and introduce the sample to the sensor cell array (i.e. detector) wherein the piezoelectric sensors are mounted. Advantageously, water quality parameters may be analyzed by detecting a change in a physical property of the sensors. Furthermore, the system may be configured such that warnings, alarms and process control commands may be displayed on a local or remote monitor and be sent to one or more prescribed recipients when certain water quality thresholds are reached.

The following describes methods of online monitoring of fluid parameters, in accordance with various embodiments.

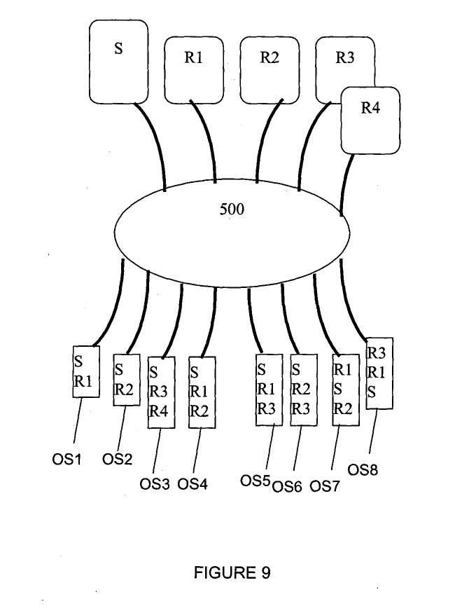

FIG. 9 illustrates a method for detecting a plurality of analytes in a single sample S. Specifically, in an embodiment, the controller 500 receives as separate inputs: a sample stream S, and first to fourth amplifying fluids R1 to R4. In an embodiment, the controller 500 forms a plurality of output streams OS1 to OS8 using the inputs. In an embodiment, each output stream contains some of the sample stream mixed with one or more of the amplifying streams. For example, OS1 contains R1, OS2 contains R2, OS3 contains R3 and R4, OS4 contains R1 and R2, OS5 contains R1 and R3, OS6 contains R2 and R3, OS7 contains R1 and R2, and OS8 contains R1 and R3. In an embodiment, each different amplifying fluid and different mixture of amplifying fluids may facilitate detection of a different analyte. Stated differently, each amplifying fluid or each amplifying mixture may amplify the interaction produced by a different detecting agent/analyte pair. Accordingly, the controller 500 may be configured to detect multiple analytes in a single sample.

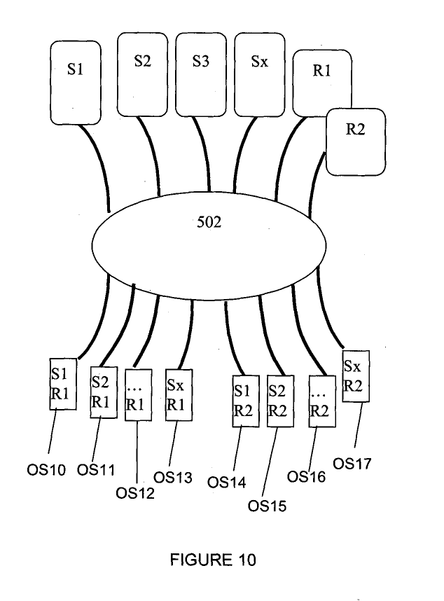

FIG. 10 illustrates a method of detecting two analytes in a plurality of different samples S1 to Sx. Specifically, in an embodiment, the controller 502 receives as separate inputs: first and second amplifying fluids R1 and R2, and multiple sample streams S1 to Sx. It is to be understood that x may be any number, for example, 5, 10, 100, 500, 1000, etc. In an embodiment, the controller 502 forms a plurality of output streams OS10 to OS17 using the inputs. In an embodiment, each output stream contains one of the sample streams and either one of the two amplifying fluids. For example, OS10 contains S1 and R1, whereas OS15 contains S2 and R2. In an embodiment, each different amplifying fluid may facilitate detection of a different analyte. Stated differently, each amplifying fluid may amplify the interaction produced by a different detecting agent/analyte pair. Accordingly, the controller 502 may be configured to detect one or more analytes in each of a plurality of samples.

In an embodiment, the system is configured to perform fingerprinting of a fluid under test. For example, the fluid may be water and the system may be employed to detect the presence of a particular species of bacteria which may render the water unfit for human consumption. Furthermore, different strains of the bacteria may be more harmful than others. Accordingly, in an embodiment, sensors of the detector may be configured in use to detect an analyte which corresponds to the bacteria. Additionally, in an embodiment, different samples of the fluid under test may be combined with different amplifying fluids. For example, different strains of the bacteria may cause a more detectable interaction in the presence of different amplifying fluids.

In use, the controller may combine each sample with a different amplifying fluid or mixture of amplifying fluids. Following which, interactions may be detected using the various output streams. The detection data obtained for all output streams may be combined and considered as a whole by the data acquirer to investigate the presence of the bacteria in the water under test. For example, the presence of the bacteria may be detected using multiple output samples and multiple different amplifying fluids and amplifying fluid mixtures. Therefore, it may be possible to confidently determine that the water under test contains the bacteria. However, this information alone may not make it possible to identify the particular bacteria strain. Since different strains of the bacteria cause more detectable interactions in the presence of a different amplifying fluid or mixture of amplifying fluids, the interaction magnitudes (i.e. levels) associated with each amplifying fluid or amplifying fluid mixture may be used to identify which strain of bacteria is present in the water under test. For example, a high magnitude detected using certain amplifying fluids and a low magnitude detected using other certain amplifying fluids may indicate one specific strain. Stated differently, multiple magnitudes may be considered together and used as a fingerprint which identifies the presence of one particular bacteria strain. Accordingly, the detection data magnitudes from multiple sensors may be used to identify the fingerprint of particular fluid components.

In an embodiment, sample streams pumped from the sampler are combined and/or split into multiple channels in the detector to form a plurality of output streams. In an embodiment, the plurality of output streams are directed to sensor cells with sensors coated with various receptors (i.e. detecting agents) to interact with target species (i.e. analytes). In an embodiment, a frequency change resulting from interactions detected at one or more of the sensors are recorded. Accordingly, direct measurement of target species or pollutants (i.e. analytes) is achieved. In an embodiment, the sensor may be additionally configured to perform ORP analysis. Accordingly, carbohydrates and amino acids may be analyzed to determine the concentration thereof, so that the concentration may be used for prediction of water quality parameters, such as, for example, COD, BOD, and TOC. Therefore, data acquired from ORP analysis may be complementary to, and correlated with, data acquired from piezoelectric sensing.

In an embodiment, stepwise sample flow with intermittent static data acquisition may be performed. In an embodiment, the sample streams are interrupted and controlled by the controller in such a way that sample streams flow through the sensor cells in a stepwise or intermittent manner. This operation creates a slow flow rate during sensing periods to ensure effective sensing. In an embodiment, data acquisition is turned on and off when the output stream is in static state and moving state, respectively. Stated differently, the system is configured such that data is only acquired from a sensor during times when the output stream being directed through the sensor is stationary, i.e. not when the output stream is flowing. Advantageously, static data acquisition improves stability of electronic data obtained from a sensor.

In an embodiment, sample dividing and cyclic sequential sensing for continuous monitoring may be performed. Specifically, it may take a time T to complete a sensing operation in respect of an output stream having a flow rate FL1. If the sensing flow rate is lower than the main fluid stream flow rate FL2, more than one sensor cells may be used to continuously monitor the complete stream. In an embodiment, the number of sensor cells N is determined by N=FL2/FL1. In an embodiment, FL1 is controlled to ensure that N is an integral number. In an embodiment, a sample stream is divided into N sequential portions and each portion is directed to a sensor cell n at time t=nT/N wherein n is 1, 2 . . . N. Accordingly, data from these N sensor cells may be combined to recover the complete data line. In an embodiment, this process is controlled by the controller and is performed in a cyclic way for continuous monitoring.

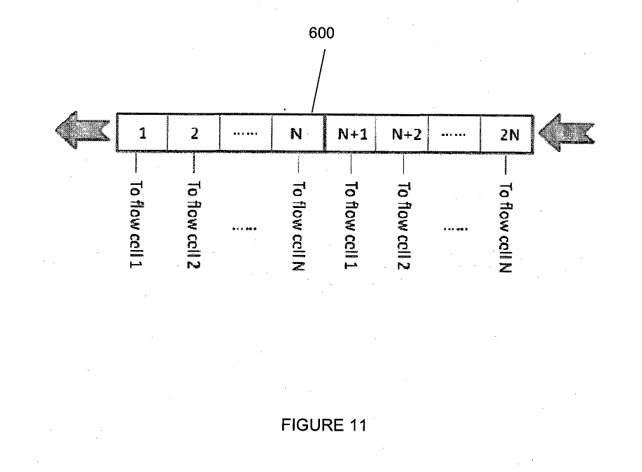

FIG. 11 illustrates the above embodiment in more detail. In an embodiment, it may take a sensor ten time units to perform one sensing operation to determine the presence of a particular analyte in an output stream. However, it may be desired that a new sensing result is provided by the system every time unit. Therefore, to meet this operational requirement, the system may be configured in use to take from a sample stream 600 a new portion (1, 2, N, N+1, N+2, 2N, where N is 10) every time unit, convert this portion into an output stream, and provide the output stream to the next one in a series of ten sensors (i.e. flow cells 1, 2, N, where N is 10). Accordingly, the output stream relating to portion 1 is provided to sensor 1, the output stream relating to portion 2 is provided to sensor 2, the output stream relating to portion 3 is provided to sensor 3, and so on until, the output stream relating to portion 10 is provided to sensor 10. At this time, ten time units have occurred since sensor 1 received its output stream; therefore, sensor 1 is ready to provide the first reading. Following this, an output stream relating to portion 11 is provided to sensor 1 to begin the cycle again. One time unit after the first reading was made sensor 2 is ready to provide the second reading. Accordingly, the system according to an embodiment may be operated to provide one new sensing result every one time unit, in spite of the fact that it takes ten time units to perform one sensing result. In an embodiment, the time taken to flush or regenerate a sensor may be accounted for to ensure that regular continuous detection is not hindered.

In various embodiments, aspects of the system may be configured to reduce bubbling of fluid, i.e. the formation of bubbles in the fluid. For example, the various tubes of the system may be configured with rounded bends, rather than sharp bends. In this way, it may be possible to reduce turbulence in the fluid and, therefore, reduce the chances of bubbles being present in the output streams when they contact the sensors. This can be an advantage because such bubbles may impede detection of interactions and, therefore, hinder the detection of analytes.

In various embodiments, a pumping device is provided to provide an intermittent flow of an output stream through a sensor cell. In an embodiment, the pumping device comprises a peristaltic pump. In an embodiment, the intermittent flow is generated by activation and deactivation of the pumping device, i.e. the pumping device is alternately switched on and off. However, in another embodiment, the pumping device includes a liquid motion switch to enhance the stability of the stepwise sample flow. For example, rather than turning on and off, the liquid motion switch may be configured to control the pumping operation of the pumping device to alternatively operate on the output stream and not operate on the output stream. Stated differently, the pumping device operates continuously; however, it may only periodically operate on the output stream. Accordingly, noise generated by the switching on and off of the pumping device may be avoided. Therefore, the stability of the electronic data signals received from the detector may be improved.

It is an advantage of various embodiments that a system is provided which allows flexible arrangements of sample chambers. For example, the system may be configured to detect a single analyte in a plurality of samples, detect a plurality of analytes in a single sample or detect a plurality of analytes in a plurality of samples. Additionally, one or more different samples may be examined at the same or different times to one or more other samples.

It is an advantage that the controller is programmable to deliver output streams comprising one or more samples and, optionally, one or more buffering fluids and/or one or more amplifying fluids. In an embodiment, the controller may operate according to a protocol which can be controlled, such as, for example, by a computing device (e.g. computing device 1000).

It is an advantage of various embodiments that online monitoring of a wide range of water quality parameters may be performed. It is an advantage of various embodiments that continuous and real-time monitoring is achieved. It is an advantage of various embodiments that multiple water quality parameters can be detected simultaneously.

It is an advantage of various embodiments that analytes may be detected in a fluid automatically. Stated differently, no skilled personnel may be required, for example, to perform wet chemistry methods. Instead, various embodiments may function automatically to collect fluid sample streams and analyse the fluid in those streams for the presence of one or more analytes.

Accordingly, detection may be performed considerably faster compared to methods utilising wet chemistry methods performed by skilled personnel.

It is an advantage of various embodiments that a multi-functional system is provided. For example, the controller can be used to generate different output streams comprising different combinations of sample streams, buffer fluids and/or amplifying fluids. Also, the controller can be used to provide each output stream to different sensors for detections of different analytes. Further, the controller can be used to provide different output streams to a corresponding sensor at different times, such that different output streams can be analysed at the same or different time to other output streams.

It is an advantage of various embodiments of the invention that the sensor system features high selectivity and sensitivity by using coating recipes. The coating recipes allow the sensor to bind to analytes and amplifying agents so that detectable, or more detectable, signals are generated. It is an advantage of various embodiments of the invention that COD, BOD, TOC, etc. can be monitored by biosensing and electrochemical sensing within the sensor system.

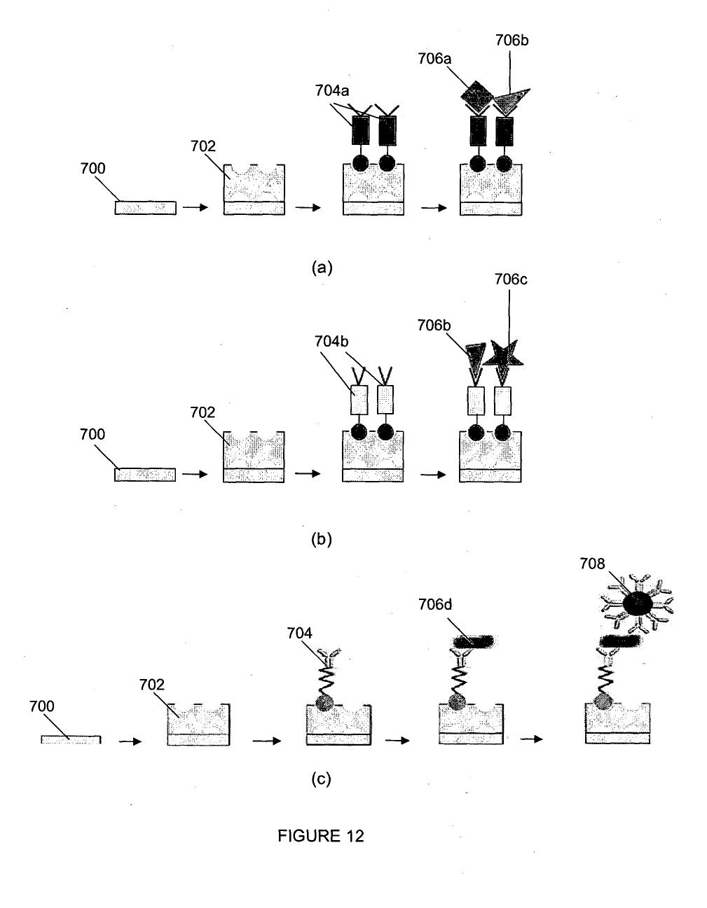

FIG. 12 illustrates examples of sensor coating recipes. In the embodiment of FIG. 12a, the sensor 700 surface is first coated with a first linking agent 702. In an embodiment, the first linking agent 702 may be avidin. In an embodiment, a blocking agent (not shown) may be used, for example, if the coverage of the linking agent 702 is less than 100%. In an embodiment, a sensing agent 704a is loaded onto the sensor 700 coated with the first linking agent 702 in order to generate a sensing surface. In an embodiment, the sensing agent 704a may be labelled with a second linking agent that is complementary to the first linking agent 702. In an embodiment, the sensing agent 704a is lectin. In an embodiment, the second linking agent is biotin. In an embodiment, the sensing surface selectively binds to an analyte upon exposure to a sample containing the analyte. In FIG. 12a, the sensing surface may be configured to bind to two different types of analyte, one type being identified as 706a (i.e. the square) and the other type being identified as 706b (i.e. the triangle). In an embodiment, the analyte 706a and/or the analyte 706b is bacteria.

FIG. 12b illustrates another example of a coating recipe, comprising a sensing agent 704b. In an embodiment, the sensing agent 704b is different from the sensing agent 704a of FIG. 12a. In an embodiment, the sensing surface of FIG. 12b may bind to the same type of analyte as the sensing surface of FIG. 12a, such as, for example, the analyte 706b (i.e. the triangle). In an embodiment, the sensing surface of FIG. 12b may bind to one or more different types of analyte as the sensing surface of FIG. 12a, such as, for example, an analyte 706c (i.e. the star). In this way, the embodiments of FIGS. 12a and 12b may illustrate how a sensor array having n (e.g. n=2) sensors is capable of detecting 2ⁿ-1 (i.e. 3) different types of analyte (i.e. the square 706a, the triangle 706b, and the star 706c).

The embodiment of FIG. 12c illustrates how the sensitivity can be further improved by adding an amplifying agent 708. In an embodiment, an analyte 706d may bind with the sensing surface, as described above. Additionally, however, the amplifying agent 708 may then bind with the analyte 706d to amplify the interaction caused by the binding of the analyte 706d with the sensing surface. In this way, detection of the analyte 706d may be improved. In an embodiment, the amplifying agent 708 may be added to a sample containing the analyte 706d. In an embodiment, the amplifying agent 708 may be a gold nanoparticle (AuNP) labelled with an antibody (Ab), a protein, or an oligonucleotide.

It is an advantage of various embodiments that no toxic chemicals are added to the fluid under test. For example, where an embodiment is used for detecting water quality, the process of testing the water does not render the water toxic or unfit for human consumption.

In an embodiment, a system for detecting one or more analytes in a fluid provides a multiple channel piezoelectric sensor system comprising: a sampler, a controller, a sensor cell array equipped with dual-crystal piezoelectric sensors plus ORP electrodes (i.e. a detector) and a data process/reporting module (i.e. a data acquirer and a notifier). An advantage of various embodiments is that the system can monitor various water quality parameters including COD, BOD and TOC. In an embodiment, monitoring of COD, BOD and TOC is done by detection of carbohydrates and amino acids using piezoelectric sensing coupled with ORP analysis. In an embodiment, continuous monitoring is achieved by using sample dividing and cyclic sequential sensing. In an embodiment, signal stability is enhanced by using dual-crystal piezoelectric sensors for noise elimination. In an embodiment, signal stability is enhanced by using stepwise sample flow with intermittent static data acquisition. In an embodiment, mathematical and statistical methods are used for noise elimination and data processing.

In an embodiment, it is to be understood that the detector may include any number of sensors. For example, the detector may include 5, 10, 100, 500, or 1000 or more sensors. Additionally, in an embodiment, each sensor may be the same; however, in another embodiment, one or more sensors may be different from one or more other sensors. For example, some sensors could include piezoelectric biosensors, whilst other sensors may include surface plasmon resonance biosensors, whilst other sensors may include electrochemical sensors. In an embodiment, one sensor may include the functionality of multiple sensors.

In an embodiment, the controller may form or generate the same number of output streams as there are sensors of the detector. Accordingly, the controller may provide a different one of its output streams to a different one of the detector's sensors. Additionally or alternatively, the controller may provide multiple output streams to the same detector sensor. Additionally or alternatively, the controller may split one or more output streams and provide each portion to a different detector sensor. In an embodiment, the number of output streams may be more than the number of detector sensors. In an embodiment, the number of output streams may be less than the number of number of detector sensors.

In some above-described embodiments, the controller is configured to control the time at which each sample stream is obtained and/or the time at which each output stream is provided to a sensor of the detector. Accordingly, the controller may be configured to hold or store fluid during its transition from a sample stream to an output stream. This may be an advantage in situations where fluids, such as, amplifying fluids, are added to the output stream. Specifically, the controller may allow the output stream to be pre-processed before it is contacted with a sensor. Stated differently, while the output stream is being held in the controller, the amplifying fluid may be acting on analytes in order that a more detectable interaction is obtained when the detecting agent is later introduced via a sensor. In an embodiment, a specific pre-processing container may be provided in the controller which is configured in use to hold fluid undergoing a preprocessing operation. In an embodiment, the pre-processing container may be one of the above-described reservoirs.

In the above-described embodiments, the fluid in which one or more analytes are detected is described in general terms. Further, in various embodiments, the fluid is water. In an embodiment, the fluid is drinking water, process water, recycled water, treated water, ground water, reservoir water and catchment water. However, it is to be understood that various embodiments are equally applicable for detecting one or more analytes in any other fluid, such as, for example, any liquid or any gas. In an embodiment, wherein the fluid is a liquid, the fluid may be an aqueous solution, a solution of organic compounds, oil, a beverage, such as, milk, a carbonated drink, a soda, a fruit drink, a fruit juice, or a body fluid, such as, blood or urin. In an embodiment, wherein the fluid is a gas, the fluid may be breathing air, oxygen, or the vapour of a liquid, outgassing from a liquid and/or a solid.

In the above-described embodiment, the various fluid streams disclosed are transported or carried using a tube. It is to be understood that the term tube is not taken to be limiting. Instead, the term tube is used to include any vessel suitable for carrying a fluid. For example, the tube could be a pipe, a conduit, a channel. Additionally, the tube may be flexible or rigid.

In the above-described embodiments, an interaction is said to occur when a detecting agent and a corresponding analyte bind together in each other presence. However, in an embodiment, an interaction may occur not when a single detecting agent and a single analyte bind together, but when two or more different detecting agents bind together with one or more analytes. In an embodiment, an interaction may occur not when a single detecting agent and single analyte bind together, but when two or more different analytes bind together with one or more detecting agents.

In an embodiment, an analyte may be an organic molecule, an inorganic molecule or a microorganism. In an embodiment, an analyte may be a contaminant or an impurity. In an embodiment, non-limiting examples of an analyte include: herbicides, pesticides, drugs, hormones, cations, anions, metal complex, particles, bacteria and viruses.

In an embodiment, a detecting agent may be an organic molecule, such as for example, an enzyme, an antibody or an organic polymer. In an embodiment, a detecting agent may be an inorganic molecule, such as, for example, a salt or an inorganic polymer. In an embodiment, a detecting agent may be a biologically sensitive agent or a chemically sensitive agent. In an embodiment, non-limiting examples of a detecting agent include: functional proteins, antibodies, oligonucleotides, and imprint polymers. In an embodiment, the detecting agent may be an antibody, a protein, an oligonucleotide or a polymer, an inorganic material, an organic substance.

In an embodiment, a flushing fluid is any fluid which can be used to remove a bound analyte from a sensor. For example, the analyte may be bound to an external coating of the sensor, wherein the coating includes a detecting agent. In an embodiment, non-limiting examples of a flushing fluid include: buffer solutions with different acidity from the buffering fluid, detergents, and organic solvents.

In an embodiment, a regeneration fluid is any fluid which can be used to form a detecting agent when it is exposed to a sensor. For example, the regeneration fluid may react with the sensor surface to form the detecting agent on the sensor surface. Alternatively, the regeneration fluid may include the detecting agent such that contacting the sensor surface with the regeneration fluid transfers the detecting agent to the sensor surface. In an embodiment, non-limiting examples of a regeneration fluid include: chemically modified functional proteins, antibodies, oligonucleotides, imprint polymers, and biotin.

In an embodiment, an amplifying fluid is any fluid which can be used to amplify an interaction caused by an analyte binding with a detecting agent. In an embodiment, the interaction may be amplified by the analyte binding to the amplifying fluid. In an embodiment, non-limiting examples of an amplifying fluid include: functional proteins, antibodies, oligonucleotides, polymers, nanoparticles, micro-beads.

In an embodiment, a buffering fluid is any fluid which can be used to maintain or set a predetermined pH in a fluid to which it is added. In an embodiment, non-limiting examples of a buffering fluid include: aqueous solutions of acetate, phosphate, 4-(2-hydroxyethyl)-1-piperazineethanesulfonic acid (HEPES), and tris(hydroxymethyl)aminomethane (Tris).

In an embodiment, an interaction is the binding of an analyte with a detecting agent. In an embodiment, the binding may be permanent or, alternatively, may be temporary. In an embodiment, the binding may include covalent binding. In an embodiment, the binding may include ionic binding. In an embodiment, the binding may include coordinative binding. In an embodiment, the binding may include metallic bonding. In an embodiment, the binding may include van der Waals forces. In an embodiment, the binding may include electrostatic binding. In an embodiment, the binding may include enzymatic binding, i.e. binding of an enzyme.

In an embodiment, an interaction is the conversion of a detecting agent caused by the presence of an analyte. In an embodiment, an interaction is the conversion of an analyte caused by the presence of a detecting agent. In an embodiment, the conversion may be temporary or, alternatively, may be permanent.

In an embodiment, a detecting agent and a corresponding analyte bind together and the interaction comprises unbinding of the corresponding analyte and at least part of the detecting agent. In an embodiment, the unbinding operation corresponds to at least one of the above-described binding operations. In an embodiment, analyte-specific molecules are immobilized on the sensor surface. The immobilized molecules are combined with substrates and signal amplification particles. In the presence of the analyte, the substrate will cleave into multiple pieces. The signal amplification particles attached on the cleaved substrates will be removed, resulting in an observable change in detection signal. Thus, through the monitoring of signal change, it is possible to determine the concentration of the analyte in the solution.

In an embodiment, the interaction comprises at least one chemical reaction, and at least one sensor detects the interaction by detecting a physical change caused by the at least one chemical reaction. In an embodiment, the chemical reaction may include an inorganic reaction, an organic reaction, a biochemical reaction.

In an embodiment, the system is provided in a portable casing so that it may be carried by a user from one fluid analysis location to another fluid analysis location. Non-limiting examples of a fluid analysis location may be a water treatment facility, a restaurant, a domestic house or a commercial building.

It will be appreciated by a person skilled in the art that numerous variations and/or modifications may be made to one or more of the above-described embodiments without departing from the spirit or scope of the invention as broadly described in the appended claims. The above-described embodiments are, therefore, to be considered in all respects to be illustrative and not restrictive.

It is also to be understood that one or more features of one embodiment may be combined with one or more features from one or more other embodiments in order to form one or more new embodiments, without departing from the scope of the appended claims.

1. A system for detecting one or more analytes in a fluid, the system comprising:
a controller adapted to obtain one or more sample streams of the fluid, the controller being configured in use to form a plurality of output streams, each output stream comprising at least part of one of the one or more sample streams; and
a detector adapted to receive from the controller the plurality of output streams and comprising a plurality of sensors, each sensor being operable to detect an interaction between a corresponding detecting agent and a corresponding analyte, the detector being configured in use to detect one or more said interactions using the plurality of output streams to determine if the fluid contains one or more said analytes.

2. The system of claim 1, wherein the corresponding detecting agent of one of the plurality of sensors is formed on said one sensor.

3. The system of claim 2, wherein the interaction detected by said one sensor comprises binding of the corresponding analyte to the corresponding detecting agent, and wherein the controller is adapted to obtain a flushing fluid configured in use to remove a bound analyte, the controller being configured in use to provide the flushing fluid to the detector to remove the bound corresponding analyte from said one sensor.

4. The system of claim 1, wherein the controller is adapted to obtain a regeneration fluid configured in use to form a detecting agent when exposed to a sensor, the controller being configured in use to provide the regeneration fluid to the detector to form the corresponding detecting agent of one of the plurality of sensors by exposing said one sensor to the regeneration fluid.

5. The system of claim 4, wherein the regeneration fluid comprises the corresponding detecting agent and exposure of said one sensor to the regeneration fluid transfers the corresponding detecting agent to said one sensor.

6. The system of claim 4, further comprising:
a regeneration fluid reservoir adapted to temporarily store the regeneration fluid before the controller obtains the regeneration fluid, the regeneration fluid reservoir being configured in use to control the temperature of the stored regeneration fluid.

7. The system of claim 1, wherein the controller is adapted to obtain at least one amplifying fluid configured in use to amplify an interaction to aid detection thereof by a sensor, the controller being configured in use to add the at least one amplifying fluid to one of the plurality of output streams.

8. The system of claim 7, further comprising:
an amplifying fluid reservoir adapted to temporarily store the at least one amplifying fluid before the controller obtains the at least one amplifying fluid, the amplifying fluid reservoir being configured in use to control the temperature of the stored at least one amplifying fluid.

9. The system of claim 1, wherein the controller is adapted to obtain a buffering fluid configured in use to maintain a predetermined pH in an output stream to which it is added, the controller being configured in use to add the buffering fluid to one of the plurality of output streams.

10. The system of claim 1, wherein one of the plurality of sensors is housed within a sensor cell having an adjustable volume for receiving a portion of one of the plurality of output streams, said one sensor being operable to detect the interaction using the portion of said one output stream received in the sensor cell.

11. The system of claim 10, wherein the controller comprises a pumping device operable to adjust the flow of said one output stream through the sensor cell.

12. The system of claim 12, wherein the pumping device is configured in use to provide an intermittent flow of said one output stream through the sensor cell.

13. The system of claim 1, wherein the controller is configured in use to provide one of the plurality of output streams to the detector at a different time to another of the plurality of output streams.

14. The system of claim 1, further comprising:
a sampler adapted to receive the fluid from a fluid source and comprising a storage unit for temporarily storing the fluid, the sampler being configured in use to form the one or more sample streams from the fluid stored in the storage unit.

15. The system of claim 12, further comprising:
a data acquirer adapted to receive electronic data from one of the plurality of sensors, wherein the electronic data indicates detection of the analyte corresponding to said one sensor.

16. The system of claim 15, wherein the data acquirer is operable to receive electronic data from said one sensor only when the pumping device flows said one output stream through the sensor cell.

17. The system of claim 15, wherein said one sensor is additionally operable to detect background noise and include in the electronic data an indication of the background noise detected at said one sensor, and wherein the data acquirer is configured in use to use the indication of the background noise to cancel background noise in the electronic data.

18. The system of claim 1, further comprising:
a notifier adapted to receive the electronic data from the data acquirer, wherein the notifier is configured in use to generate a notification in dependence on the received electronic data.

19. The system of claim 1, wherein the corresponding detecting agent of one of the plurality of sensors is different to the corresponding detecting agent of another of the plurality of sensors, and wherein the interaction relating to said one sensor indicates the presence of a different corresponding analyte to the interaction relating to said other sensor.

20. The system of claim 1, wherein one of the plurality of sensors is operable to detect a level of interaction using the corresponding detecting agent, wherein the level of interaction indicates an amount of the corresponding analyte which is present.

21. The system of claim 1, wherein the interaction comprises binding together of the corresponding detecting agent and the corresponding analyte, and wherein at least one sensor detects the interaction by detecting a physical change caused by the binding operation.

22. The system of claim 1, wherein the corresponding detecting agent and the corresponding analyte bind together and the interaction comprises unbinding of the corresponding analyte and at least part of the corresponding detecting agent, and wherein at least one sensor detects the interaction by detecting a physical change caused by the unbinding operation.

23. The system of claim 1, wherein the interaction comprises at least one chemical reaction, and wherein at least one sensor detects the interaction by detecting a physical change caused by the at least one chemical reaction.

24. The system of claim 21, wherein the physical change is at least one of the following group: a change in mass, a change in electrical resistivity, a change in electrical conductivity, a change in refractive index, a change in electric current, a change in electric potential.

25. The system of claim 24, wherein the physical change occurs to the at least one sensor.

26. The system of claim 1, wherein the detecting agent is one of the following group: an antibody, a protein, an oligonucleotide, a polymer, an inorganic material, an organic substance.

27. The system of claim 1, wherein one of the plurality of sensors is at least one of the following group: a piezoelectric sensor, an electrochemical sensor, an electrochemical quartz crystal microbalance sensor, a surface plasmon resonance sensor, an oxidation reduction potential sensor.

28. A method for detecting one or more analytes in a fluid, the method comprising:
obtaining one or more sample streams of the fluid,
forming a plurality of output streams, each output stream comprising at least part of one of the one or more sample streams,
detecting one or more interactions using the plurality of output streams and a plurality of sensors, wherein each sensor is operable to detect an interaction between a corresponding detecting agent and a corresponding analyte, and
determining if the fluid contains one or more said analytes based on the one or more detected interactions.

29. The method of claim 28, further comprising forming the corresponding detecting agent of one of the plurality of sensors on said one sensor.

30. The method of claim 29, wherein the interaction detected by said one sensor comprises binding of the corresponding analyte to the corresponding detecting agent, and the method further comprises removing the bound corresponding analyte from said one sensor by exposing said one sensor to a flushing fluid configured in use to remove a bound analyte.

31. The method of claim 28, further comprising forming a corresponding detecting agent of one of the plurality of sensors by exposing said one sensor to a regeneration fluid.

32. The method of claim 31, wherein the regeneration fluid comprises the corresponding detecting agent and exposure of said one sensor to the regeneration fluid transfers the corresponding detecting agent to said one sensor.

33. The method of claim 31, further comprising:
temporarily storing the regeneration fluid before exposing said one sensor to the regeneration fluid; and
controlling the temperature of the regeneration fluid whilst it is being temporarily stored.

34. The method of claim 28, further comprising adding at least one amplifying fluid to one of the plurality of output streams, the at least one amplifying fluid being configured in use to amplify an interaction to aid detection thereof by a sensor.

35. The method of claim 34, further comprising:
temporarily storing the at least one amplifying fluid before adding the at least one amplifying fluid to said one output stream; and
controlling the temperature of the at least one amplifying fluid whilst it is being temporarily stored.

36. The method of claim 28, further comprising adding a buffering fluid to one of the plurality of output streams, the buffering fluid being configured in use to maintain a predetermined pH in said one output stream.

37. The method of claim 28, further comprising adjusting a flow of at least one output stream through at least one of the plurality of sensors.

38. The method of claim 37, further comprising adjusting the flow to be intermittent.

39. The method of claim 28, wherein said step of detecting one or more interactions comprises using one of the plurality of output streams at a different time to another of the plurality of output streams.

40. The method of claim 28, further comprising:
obtaining the fluid from a fluid source; and
temporarily storing the obtained fluid before forming the one or more sample streams of the fluid from the temporarily stored fluid.

41. The method of claim 38, further comprising:
receiving electronic data from one of the plurality of sensors, wherein the electronic data indicates detection of the analyte corresponding to said one sensor.

42. The method of claim 41, wherein electronic data is received from said one sensor only when said one output stream flows through said one sensor.

43. The method of claim 41, wherein said one sensor is additionally operable to detect background noise and include in the electronic data an indication of the background noise detected at said one sensor; and the method further comprises cancelling background noise in the electronic data using the indication of the background noise.

44. The method of claim 41, further comprising: generating a notification in dependence on the received electronic data.

45. The method of claim 28, wherein the corresponding detecting agent of one of the plurality of sensors is different to the corresponding detecting agent of another of the plurality of sensors, and wherein the interaction relating to said one sensor indicates the presence of a different corresponding analyte to the interaction relating to said other sensor.

46. The method of claim 28, further comprising detecting a level of interaction using one of the plurality of sensors, wherein the level of interaction indicates an amount of the corresponding analyte which is present.

47. The method of claim 28, wherein the interaction comprises binding together of the corresponding detecting agent and the corresponding analyte; and the method further comprises detecting the interaction by detecting a physical change caused by the binding operation.

48. The method of claim 28, wherein the corresponding detecting agent and the corresponding analyte bind together and the interaction comprises unbinding of the corresponding analyte and at least part of the corresponding detecting agent; and the method further comprises detecting the interaction by detecting a physical change caused by the unbinding operation.

49. The method of claim 28, wherein the interaction comprises at least one chemical reaction; and the method further comprises detecting the interaction by detecting a physical change caused by the at least one chemical reaction

50. The method of claim 47, wherein the physical change is at least one of the following group: a change in mass, a change in electrical resistivity, a change in electrical conductivity, a change in refractive index, a change in electric current, a change in electric potential.

51. The method of claim 50, wherein the physical change occurs to the corresponding sensor.

52. The method of claim 28, wherein the detecting agent is one of the following group: an antibody, a protein, an oligonucleotide, a polymer, an inorganic material, an organic substance.

53. The method of claim 28, wherein one of the plurality of sensors is at least one of the following group: a piezoelectric sensor, an electrochemical sensor, an electrochemical quartz crystal microbalance sensor, a surface plasmon resonance sensor, an oxidation reduction potential sensor.
